(19) United States
(12) Patent Application Publication
(10) Pub. No.: US 2022/0229042 A1
(43) Pub. Date: Jul. 21, 2022

(54) BIOINSTRUMENTATION APPARATUS AND METHOD

(71) Applicant: Taiwan RedEye Biomedical Inc., Hsinchu City (TW)

(72) Inventors: Shuo-Ting Yan, Hsinchu City (TW); I-Hua Wang, Hsinchu City (TW); Chen-Chung Chang, Hsinchu City (TW)

(73) Assignee: Taiwan RedEye Biomedical Inc., Hsinchu City (TW)

(21) Appl. No.: 17/154,018

(22) Filed: Jan. 21, 2021

(51) Int. Cl. G01N 33/49; G01N 21/65; E03D 9/00; G01N 21/49; G01N 21/3577

(52) U.S. Cl. CPC G01N 33/491; G01N 21/65; G01N 21/3577; G01N 21/49; E03D 9/00

(57) ABSTRACT

Disclosed is a bioinstrumentation apparatus including a main unit, a detection portion, a light source, and an optics sensor. The bioinstrumentation apparatus is a kind of home health care device for testing Triglycerides automatically without requiring any chemical reagent or blood specimen.

### BACKGROUND OF THE INVENTION

### Technical Field

The present invention relates to a bioinstrumentation apparatus and method, and more particularly relates to the bioinstrumentation apparatus and method applied to a toilet seat of a toilet.

### Description of the Related Art

Triglycerides (TG) in blood are also taken as a synonym of neutral fats, and high triglycerides in blood are usually a sign for the high risk of atherosclerosis, cardiovascular disease and stroke.

For the TG level of adults, the normal value is less than 150, the marginal high value is 150˜199, the high risk value is 200˜499, and the ultra-high risk value is greater than 500. In general, blood tests are required to measure one's TG level, and a conventional blood test technique generally requires fasting of 8˜10 hours and an invasive blood test. In addition, the test needs to be carried out in a professional medical institute and the test results can only be obtained by biochemical analysis, so the conventional test is extremely lack of immediacy and impossible to monitor the triglycerides in real time in order to achieve the effect of reducing the possibility of diseases.

Although most conventional blood tests often require fasting before the test, the concentration of triglycerides after diet will increase significantly, and not all cases of fasting are present when determining a cardiovascular disease or stroke, so that a random detection of triglycerides without the limited condition of fasting has a certain degree of medical significance.

Therefore, how to satisfy the bioinstrumentation requirements for rapidity and immediacy while taking both simplicity and non-invasiveness into account demands immediate attentions and feasible solutions.

### SUMMARY OF THE INVENTION

It is a primary objective of the present invention to overcome the aforementioned drawbacks of the prior art by providing a bioinstrumentation apparatus installed to a toilet for testing a living organism, and the bioinstrumentation apparatus comprises: a main unit including an operation control unit; and a detection portion, installed in a toilet seat of the toilet, and the detection portion includes a light source electrically coupled to the operation control unit; wherein the operation control unit controls the light source to emit an emitted light to the living organism to generate and enter a scattered light into an optical sensor, and after receiving the scattered light, the optical sensor sends a sense signal to the operation control unit, and the operation control unit determines the sense signal to output a test result.

The present invention further provides a bioinstrumentation method implemented to a toilet and applied in a bioinstrumentation apparatus for testing a living organism, and the bioinstrumentation apparatus comprises a main unit and a detection portion, and the bioinstrumentation apparatus is mounted onto the toilet by a mounting portion, and the main unit comprises an operation control unit, and the detection portion is installed in a toilet seat of the toilet and comprises a light source electrically coupled to the operation control unit; and the bioinstrumentation method comprises the steps of: receiving a start testing instruction by an input unit; driving a light source electrically coupled to the operation control unit to emit an emitted light to the living organism; generating and entering a scattered light into an optical sensor after emitting the emitted light to the living organism, and receiving a sense signal from the optical sensor by the operation control unit; and determining the sense signal and sending a test result to a display unit by the operation control unit.

The present invention adopts a non-invasive testing method to achieve an instant testing purpose without the needs of drawing blood, adding biochemical agent, fasting, and high specification of toilets as used in professional medical institutes, while satisfying the requirements for rapidity, immediacy, simplicity, and non-invasiveness.

The above and other objects and technical characteristics of the present invention will become apparent from the following detailed description taken with the accompanying drawings.

### BRIEF DESCRIPTION OF THE DRAWINGS

FIG. 1 is a side view of a bioinstrumentation apparatus installed to a toilet in accordance with a first preferred embodiment of the present invention;

FIG. 2 is a bottom view of the bioinstrumentation apparatus installed to the toilet in accordance with the first preferred embodiment of the present invention;

FIG. 3 is a structural block diagram of the bioinstrumentation apparatus installed to the toilet in accordance with the first preferred embodiment of the present invention;

FIG. 4 is a schematic view of a skin surface;

FIG. 5 is a schematic view of an emitted light projected onto living organisms with different triglycerides contents and a scattered light in accordance with a preferred embodiment of the present invention;

FIG. 6 is a comparison chart of sense signals versus conventional blood testing results in accordance with a preferred embodiment of the present invention;

FIG. 7 is a structural block diagram of the bioinstrumentation apparatus installed to the toilet in accordance with a second preferred embodiment of the present invention;

FIG. 8 is a structural block diagram of the bioinstrumentation apparatus installed to the toilet in accordance with a third preferred embodiment of the present invention;

FIG. 9 is a schematic view showing a light transmission in accordance with a third preferred embodiment of the present invention; and

FIG. 10 is a flow chart of a bioinstrumentation method of the present invention.

### DESCRIPTION OF THE PREFERRED EMBODIMENTS

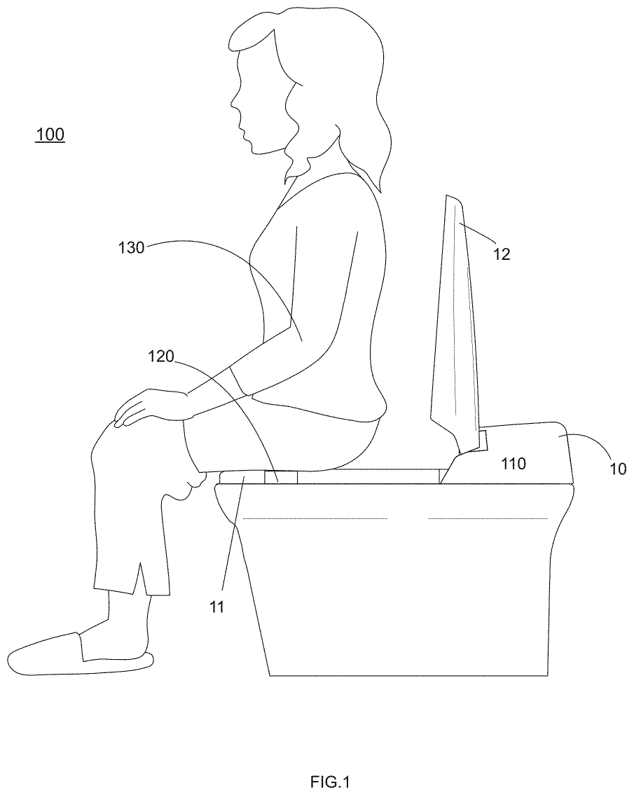

With reference to FIGS. 1˜3 for a side view, a bottom view, and a structural block diagram of a bioinstrumentation apparatus 100 installed to a toilet 10 in accordance with the first preferred embodiment of the present invention respectively, the toilet 10 comprises a toilet 11 and a toilet seat 12. The apparatus 100 comprises a main unit 110 and a detection portion 120. The main unit 110 is electrically coupled to the detection portion 120. Preferably, the main unit 110 is installed outside the toilet 10; the detection portion 120 is embedded in the toilet seat 12, so that a living organism 20 (such as a human thigh) can be in a direct contact when going to the toilet.

In a different embodiment, the present invention can set, fix or install the bioinstrumentation apparatus 100 (or the main unit 110) to the top or a side of the toilet (cover) by a mounting portion by means of a clamping suspending, attaching, pasting, binding, socketing, latching, buckling, magnetic, riveting, screwing or locking method, and each of the aforementioned methods uses a clip, an adhesive, a strap, a screw, a nut, a snap, a magnet, a rivet, or a suction disc, etc. This invention does not impose restrictions on various changes of the mounting portion, and any method capable of achieving a stable installation is applicable for the present invention.

In FIG. 3, the main unit 110 comprises an operation control unit 111 and a power supply 112. A detection portion 120 comprises a light source 121 and an optical sensor 122. The operation control unit 111 is electrically coupled to the power supply 112, and the operation control unit 111 is also electrically coupled to the light source 121 and the optical sensor 122 of the detection portion 120. Preferably, the toilet seat 11 comprises a transparent window 140, and both of the light emitting element 121 and the sensing element 122 are in a directly contact with the living organism 130 through the transparent window 140. The thickness of the transparent window 140 plus the distance from the light emitting element 121 to the transparent window 140 is smaller than 5 mm to avoid errors of the detection.

In the present invention, the operation control unit such as MCU, CPU, etc. is a unit with calculation, processing, control, and/or analysis functions. The power supply such as a battery (including a replaceable battery and a rechargeable battery) or an external AC power supply. If the power supply is an external AC power supply, the power supply will comprise a DC/AC conversion circuit. The input unit such as a physical button, a voice control module, or an LCD touch display, etc. is provided for receiving a test instruction or a control for adjusting the instruction of a support portion. If the input unit is the LCD touch display, the input unit will further comprise a user interface. The input unit has options for different testing objects (including blood, uric acid, urine protein, and urine sugar). The display unit such as an LCD touch display or an LED light signal, etc. is provided for displaying the test result and information. The light source such as an LED light source or laser, etc. has a controller capable of exciting beams of various wavelengths. The optical sensor such as a spectrophotometer, a photo-diode, a complementary metal oxide semiconductor (CMOS) sensor) or a charge coupled device (CCD) sensor has a controller. It is noteworthy that the aforementioned examples are intended for illustrating the present invention, but not for limiting the invention.

In this preferred embodiment, the bioinstrumentation apparatus 100 starts operating after receiving a pressure or touch signal, (such as the living organism 20 sitting on the toilet seat 12) and sends a test result to a computer or a mobile device coupled to the bioinstrumentation apparatus 100 after the operation ends. In the meantime, the computer or mobile device starts the operation of the bioinstrumentation apparatus 100. It is noteworthy that the present invention is not limited to the aforementioned arrangements.

In another embodiment, the main unit can be combined with an (external) electric bidet toilet which may even be able to supply power to the toilet. Further, the main unit can be integrated with the electric bidet toilet in order to share the operation control unit, the input unit, the display unit, and the power supply.

The bioinstrumentation apparatus of the present invention is used for performing a bioinstrumentation. In the following preferred embodiments of the present invention, a scatter light generated by a certain quantity of triglycerides in skin is tested. In other different embodiment, the apparatus of the present invention is used for performing different bioinstrumentations. In different embodiments, the testing principle of the bioinstrumentation makes use of the characteristics of adsorption, fluorescence, scattering or Raman spectroscopy, but the present invention is not limited to these testing principles only.

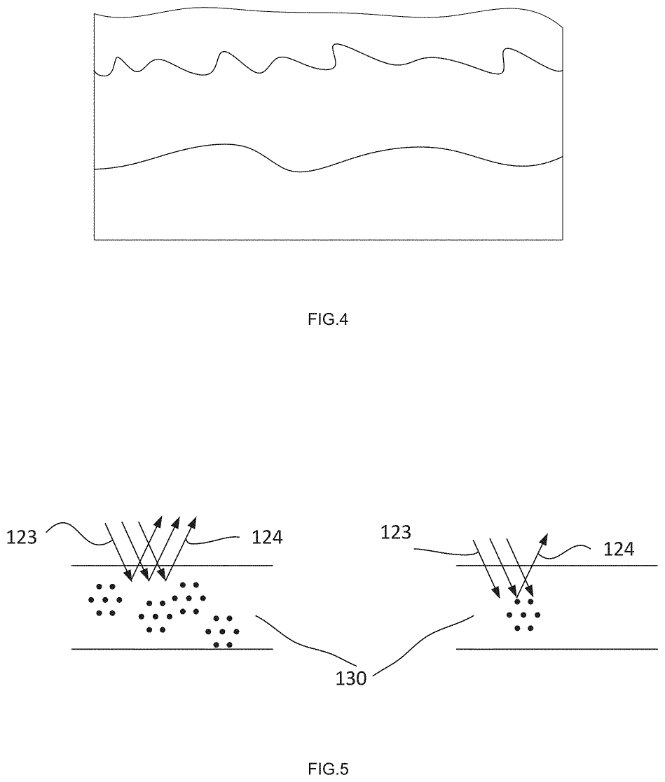

With reference to FIG. 4 for the schematic view showing the structure of a skin surface including an epidermis, a dermis) and a subcutaneous tissue arranged from top to bottom of the skin surface. Light with too short wavelength cannot penetrate through the skin effectively and light with too long wavelength will penetrate through the skin directly, so that selecting a light with appropriate wavelength as the emitted light 123 is an important technical means to complete the present invention. In order to generate and measure the scattered light 124 capable of penetrating through the skin, the operation control unit 111 controls the light source 121 to emit the emitted light 123 of a specific wavelength (500˜1200 nm), and the emitted light 123 is incident on the living organism 130 to generate and enter the scattered light 124 into the optical sensor 122. After receiving the scattered light 124, the optical sensor 122 generates a sense signal 125 which is sent to the operation control unit 111. Preferably, the emitted light 123 has wavelength with a range of 700˜1100 nm.

With reference to FIG. 5 for the schematic view of the emitted light 123 that is incident on living organisms 130 of different triglycerides contents and their scattered light 124 so generated in accordance with a preferred embodiment of the present invention, the black dots in the figure are triglycerides, and the triglycerides level of the living organism 130 on the left is obviously higher than the triglycerides level of the living organism 130 on the right. Correspondingly, under the irradiation of the emitted light 123 of the same intensity (wherein intensity is equivalent to quantity), the scattered light 124 generated by the living organism 130 on the left is obviously stronger than the scattered light 124 generated by the living organism 130 on the right, and the sense signal 125 is also stronger correspondingly.

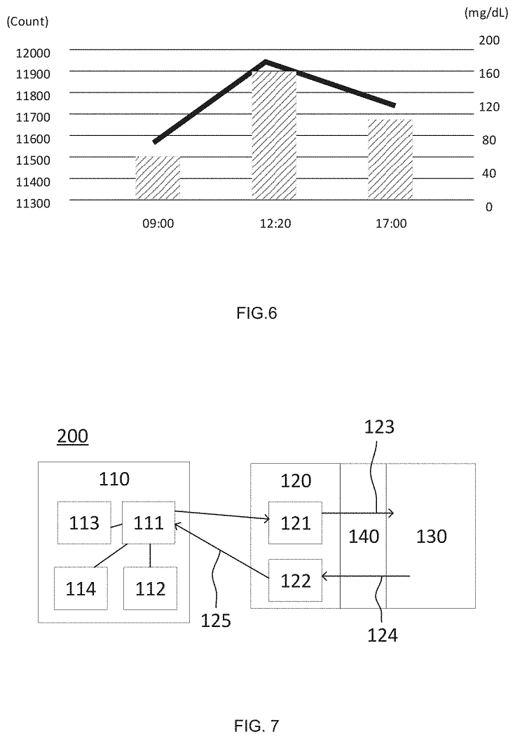

With reference to FIG. 6 for the comparison chart of sense signals 125 versus conventional blood testing results in accordance with a preferred embodiment of the present invention, the solid line in the figure indicates the data (wherein the unit on the right is mg/dL) of the conventional blood test; the bar chart adopts the data of the bioinstrumentation apparatus 100 of the present invention (the unit on the left is count; wherein the greater the count, the stronger the intensity of the triglycerides). In FIG. 6, the present invention has the same trend as the conventional blood test, indicating that the level of triglycerides in the blood test has the same trend as the sensing signal 125 of the biological body 130 (such as a thigh skin) collected by the bioinstrumentation apparatus 100 of the present invention. After receiving the sense signal 125, the operation control unit 111 determines the sense signal 125 (wherein the optical sensor 122 receives a signal related to the amount of the scattered light 124 in a preferred embodiment of this invention), and the figure shows that the intensity of signal and the value of triglycerides obtained from the conventional blood test have a positive correlation. Therefore, the conversion between the intensity of the sense signal 125 and the value of triglycerides obtained from the conventional blood test can be used to learn about the signal intensity corresponding to the numerical value of triglycerides.

With reference to FIG. 7 for the structural block diagram of a bioinstrumentation apparatus 200 installed to the toilet 10 in accordance with the second preferred embodiment of the present invention, the difference between the bioinstrumentation apparatus 200 of the second preferred embodiment and the bioinstrumentation apparatus 100 of the first preferred embodiment will be described below, but their same technical characteristics will not be repeated. The difference between the first and second preferred embodiments resides on that the main unit of the second preferred embodiment further comprises an input unit 113 and a display unit 114. The operation control unit 111 is also electrically coupled to the input unit 113 and the display unit 114, so that the input unit 113 can receive the instruction to start the test and the display unit 114 can display the test result.

In other embodiments, the main unit may be combined with an (external) electric bidet toilet, and the electric bidet toilet can even be used to supply power. Further, the main unit may be integrated with the electric bidet toilet to share the operation control unit, the input unit, the display unit and the power supply.

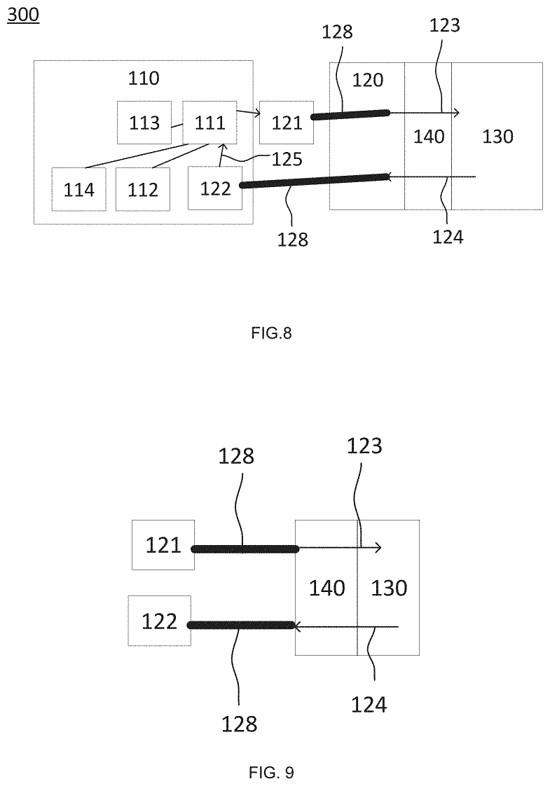

With reference to FIG. 8 for the structural block diagram of a bioinstrumentation apparatus 300 installed to the toilet 10 in accordance with the second preferred embodiment of the present invention, the difference between the bioinstrumentation apparatus 300 of the third preferred embodiment and the bioinstrumentation apparatus 200 of the second preferred embodiment will be described below, but their same technical characteristics will not be repeated. The difference between the second and third preferred embodiments resides on that the bioinstrumentation apparatus 300 of the third preferred embodiment further comprises two light pipes 128, and the light source 121 and the optical sensor 122 are installed in the main unit 110 in this embodiment. In other words, the detection portion only has two light pipes 128 left (one for transmitting the emitted light 123 and the other one for transmitting the scattered light 124). This arrangement can simplify the production process because the main components are in the main unit 110, so that the manufacturing procedure can be simplified to a certain extent. However, the invention is not limited to such arrangement only, but the light source 121 and the optical sensor 122 can be installed in the main unit 110 and the detection portion 120 respectively if necessary, and the required light pipes can be installed correspondingly.

With reference to FIG. 9 for the schematic view of light transmission in accordance with the third preferred embodiment of the present invention, the emitted light 123 is emitted by the light source 121 and passed through the light pipe 128 and the transparent window 140, and finally entered into the living organism 130. The emitted light 123 is in contact with the triglycerides in the living organism 130, and the scattered light 124 is generated and passed through the transparent window 140 and the light pipe 128 and entered into the optical sensor 122.

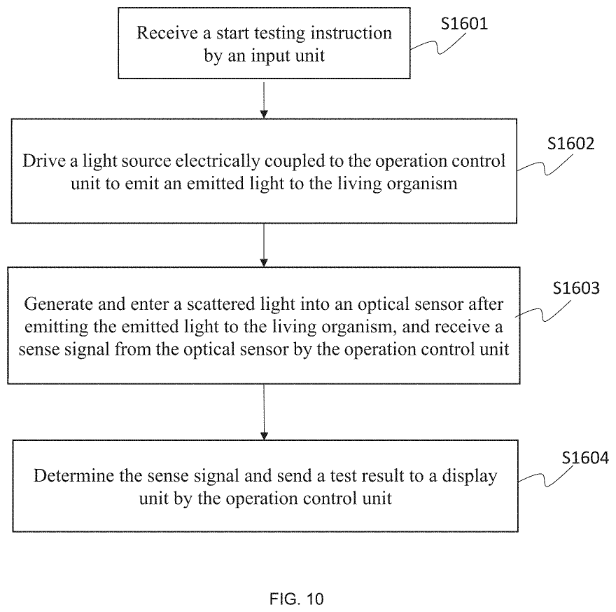

With reference to FIG. 10 for the flow chart of a bioinstrumentation method of the present invention, the bioinstrumentation method is provided for performing a bioinstrumentation, and an embodiment of testing the triglycerides of a living organism 130 is used to illustrate the present invention. In different embodiments, the testing principles of the bioinstrumentation include but not limited to using the characteristics of adsorption, fluorescence, scattering or Raman spectroscopy.

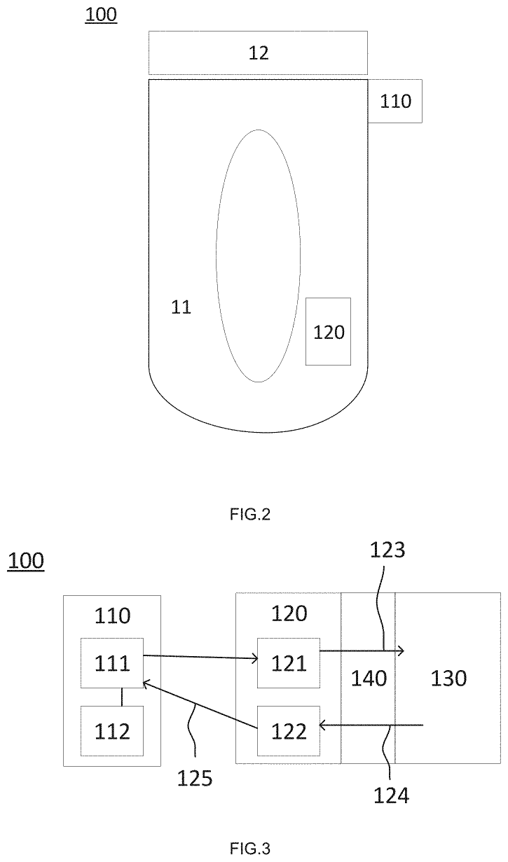

The bioinstrumentation method of the present invention is applied to the bioinstrumentation apparatus, and the related content of the bioinstrumentation apparatus of a preferred embodiment of the present invention has been described above and illustrated in FIGS. 1 to 9, and thus will not be repeated. The bioinstrumentation method comprises the following steps.

Step S1601: An input unit 113 receives a start testing instruction. Specifically, the input unit 113 receives the start testing instruction inputted by a user and then starts testing. The input unit 113 such as a physical button, a voice control module or an LCD touch display, etc. is provided for the user to input the start testing instruction or control an adjustable support portion to carry out an adjusting instruction. If the input unit 113 is the LCD touch display, then the input unit 113 will further comprise a user interface.

Step S1602: A light source 121 electrically coupled to the operation control unit 111 is driven to emit an emitted light 123 to the living organism 130. After the start testing instruction is received by the input unit 113, a start signal is generated, so that the operation control unit 111 controls the light source 121 to emit an emitted light 123 of a specific wavelength (as described above).

Step S1603: After the emitted light 123 is emitted to the living organism 130, a scattered light 124 is generated and entered into an optical sensor 122, and the operation control unit 111 receives a sense signal 125 transmitted from the optical sensor 122.

Step S1604: The operation control unit 111 determines the sense signal 125, and then sends a test result to a display unit 114.

In the aforementioned embodiment of the present invention, the basic testing principle is to measure the scattered light of triglycerides, but in fact the principle should not be limited to the scattering caused by the triglycerides. For example, the measurement of the characteristics of fluorescence, scattering, and Raman spectroscopy for the general spectrum of skin can be used as a basis for detecting the amount of triglycerides in superficial skin.

In summation, the present invention provides a bioinstrumentation apparatus (which is a home health care apparatus) and a bioinstrumentation method capable of testing triglycerides automatically without requiring the addition of any biological/chemical agent, diluent and test paper, the collection of any excrement specimen, or any blood collection procedure, which are suitable for the general public to operate at home. The non-invasive method is used to test whether the triglycerides are abnormal and capable of reducing the discomfort caused by the conventional blood test to achieve the purpose of bioinstrumentation of the present invention.

In summation of the description above, persons having ordinary skill in the art understand that the present invention surely achieves the aforementioned objectives and complies with patent application requirements, and thus is duly filed for patent application. While the invention is described with reference to certain illustrated embodiments, it is to be understood that there is no intent to limit the invention to those embodiments. On the contrary, the aim is to cover all modifications, alternatives and equivalents falling within the spirit and scope of the invention as defined by the appended claims.

What is claimed is:

1. A bioinstrumentation apparatus, installed onto a toilet, for testing a living organism, and the bioinstrumentation apparatus comprising:
a main unit, comprising an operation control unit; and
a detection portion, installed into a toilet seat of the toilet,
wherein, the operation control unit controls a light source electrically coupled thereto to emit an emitted light to the living organism to generate and enter a scattered light into an optical sensor, and then the optical sensor sends a sense signal to the operation control unit electrically coupled thereto, and the operation control unit determines the sense signal to output a test result.

2. The bioinstrumentation apparatus of claim 1, wherein the main unit further comprises an input unit and a display unit, both electrically coupled to the operation control unit, and the input unit is provided for inputting a test instruction, and the operation control unit controls the display unit to display the test result.

3. The bioinstrumentation apparatus of claim 1, wherein the optical sensor is included in the detection portion or the main unit.

4. The bioinstrumentation apparatus of claim 1, wherein the light source is included in the detection portion or the main unit.

5. The bioinstrumentation apparatus of claim 1, wherein the bioinstrumentation is a test of triglycerides.

6. The bioinstrumentation apparatus of claim 1, wherein the bioinstrumentation uses the characteristics of adsorption, fluorescence, scattering or Raman spectroscopy as a testing principle.

7. The bioinstrumentation apparatus of claim 1, wherein the main unit is installed onto the toilet by a clamping, suspending, attaching, pasting, binding, socketing, latching, buckling, magnetic, riveting, screwing or locking method.

8. The bioinstrumentation apparatus of claim 1, wherein the emitted light has a wavelength of 500˜1200 nanometers.

9. The bioinstrumentation apparatus of claim 1, wherein the emitted light has a wavelength of 700˜1100 nanometers.

10. A bioinstrumentation method, implemented to a toilet, and applied in a bioinstrumentation apparatus, for testing a living organism, and the bioinstrumentation apparatus comprising a main unit and a detection portion, and the bioinstrumentation apparatus being mounted onto the toilet by a mounting portion, and the main unit comprising an operation control unit, and the detection portion being installed in a toilet seat of the toilet; the bioinstrumentation method comprising the steps of:
receiving a start testing instruction by an input unit;
driving a light source electrically coupled to the operation control unit to emit an emitted light to the living organism;
generating and entering a scattered light into an optical sensor after emitting the emitted light to the living organism, and receiving a sense signal from the optical sensor by the operation control unit; and
determining the sense signal and sending a test result to a display unit by the operation control unit.
